(19) United States
(12) Patent Application Publication
(10) Pub. No.: US 2013/0146069 A1
(43) Pub. Date: Jun. 13, 2013

(54) Intubation Tube Oral Appliance System

(71) Applicant: JHV ENTERPRISES LLC, Horseshoe Bend, ID (US)

(72) Inventors: Jason R. Van Wagenen, Thornton, CO (US); Holly D. Carlson, Horseshoe Bend, ID (US)

(73) Assignee: JHV ENTERPRISES LLC, Horseshoe Bend, ID (US)

(21) Appl. No.: 13/713,865

(22) Filed: Dec. 13, 2012

Related U.S. Application Data
(63) Continuation-in-part of application No. 13/324,151, filed on Dec. 13, 2011.

(51) Int. Cl. A61M 16/04; A61C 5/14

(52) U.S. Cl. CPC A61M 16/0497; A61C 5/14
USPC 128/862

(57) ABSTRACT

An intubation tube oral appliance system for providing a semi-customized support for an intubation tube that protects the intubation tube and facilitates the delivery of oral hygiene materials to reduce the incidences of ventilator associated pneumonia (VAP). The intubation tube oral appliance system generally includes a tube support, a first tray extending from the tube support and a second tray extending from the tube support. The tube support is formed to receive an intubation tube and a strap or securing member secures the intubation tube to the tube support. The first tray and the second tray include a thermoplastic layer that is formed to the ventilation patient's teeth. An antibacterial solution and/or fluoride solution is placed within the first tray and second tray prior to insertion into the ventilation patient's mouth to reduce dental plaque and VAP.

### CROSS REFERENCE TO RELATED APPLICATIONS

I hereby claim benefit under Title 35, United States Code, Section 120 of U.S. patent application Ser. No. 13/324,151 filed Dec. 13, 2011. This application is a continuation-in-part of the Ser. No. 13/324,151 application. The Ser. No. 13/324,151 application is currently pending. The Ser. No. 13/324,151 application is hereby incorporated by reference into this application.

### STATEMENT REGARDING FEDERALLY SPONSORED RESEARCH OR DEVELOPMENT

Not applicable to this application.

### BACKGROUND OF THE INVENTION

1. Field of the Invention

The present invention relates generally to an intubation tube support devices and more specifically it relates to an intubation tube oral appliance system for providing a semi-customized support for an intubation tube that protects the intubation tube and facilitates the delivery of oral hygiene materials to reduce the incidences of ventilator associated pneumonia (VAP).

2. Description of the Related Art

Any discussion of the related art throughout the specification should in no way be considered as an admission that such related art is widely known or forms part of common general knowledge in the field.

Ventilator associated pneumonia (VAP) is an infection of the airways commonly associated with ventilator use. Unfortunately, VAP is a significant cause of death involving hospital acquired infections. Dental plaque develops in ventilated patients because of the lack of mechanical chewing and the absence of saliva. The dental plaque is colonized by respiratory pathogens in ventilated patients resulting in VAP. Daily oral care for a ventilated patient is important to prevent VAP by reducing the dental plaque that builds up. The current protocol in hospitals is to have a plurality of toothbrushes color coded for the time of day when the brushing should occur (typically once every two hours). Unfortunately, it is difficult and labor intensive to brush the teeth of a ventilation patient. Another problem with conventional oral care for ventilation patients is the increased possibility of tube disruption because of the periodic cleanings.

Because of the inherent problems with the related art, there is a need for a new and improved intubation tube oral appliance system for providing a semi-customized support for an intubation tube that protects the intubation tube and facilitates the delivery of oral hygiene materials to reduce the incidences of ventilator associated pneumonia (VAP).

### BRIEF SUMMARY OF THE INVENTION

The invention generally relates to an intubation tube support devices which includes a tube support, a first tray extending from the tube support and a second tray extending from the tube support. The tube support is formed to receive an intubation tube and a strap secures the intubation tube to the tube support. The first tray and the second tray include a thermoplastic layer that is formed to the ventilation patient's teeth. An antibacterial solution and/or fluoride solution is placed within the first tray and second tray prior to insertion into the ventilation patient's mouth to reduce dental plaque and VAP.

There has thus been outlined, rather broadly, some of the features of the invention in order that the detailed description thereof may be better understood, and in order that the present contribution to the art may be better appreciated. There are additional features of the invention that will be described hereinafter and that will form the subject matter of the claims appended hereto. In this respect, before explaining at least one embodiment of the invention in detail, it is to be understood that the invention is not limited in its application to the details of construction or to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

### BRIEF DESCRIPTION OF THE DRAWINGS

Various other objects, features and attendant advantages of the present invention will become fully appreciated as the same becomes better understood when considered in conjunction with the accompanying drawings, in which like reference characters designate the same or similar parts throughout the several views, and wherein:

FIG. 1 is an upper perspective view of the present invention.

FIG. 2 is an upper perspective view of the present invention with an intubation tube attached within the present invention.

FIG. 3 is a front end view of the present invention.

FIG. 4 is a rear end view of the present invention.

FIG. 5 is a top view of the present invention.

FIG. 6 is a bottom view of the present invention.

FIG. 7 is a left side view of the present invention.

FIG. 8 is a cross sectional view taken along line 8-8 of FIG. 2.

FIG. 9 is an upper perspective view of the present invention positioned within the mouth of a patient.

FIG. 10 is an upper perspective view of a first alternative embodiment of the present invention utilizing a head strap.

FIG. 11 is an upper perspective view of a second alternative embodiment of the present invention.

FIG. 12 an upper perspective view of the second alternative embodiment with an intubation tube attached within.

FIG. 13 is a front end view of the second alternative embodiment securing an intubation tube.

FIG. 14 is a rear end view of the second alternative embodiment securing an intubation tube.

FIG. 15 is a top view of the second alternative embodiment.

FIG. 16 is a bottom view of the second alternative embodiment.

FIG. 17 is a left side view of the second alternative embodiment.

FIG. 18 is a cross sectional view taken along line 18-18 of FIG. 12.

FIG. 19 is a cross sectional view taken along line 19-19 of FIG. 12.

### DETAILED DESCRIPTION OF THE INVENTION

### A. Overview

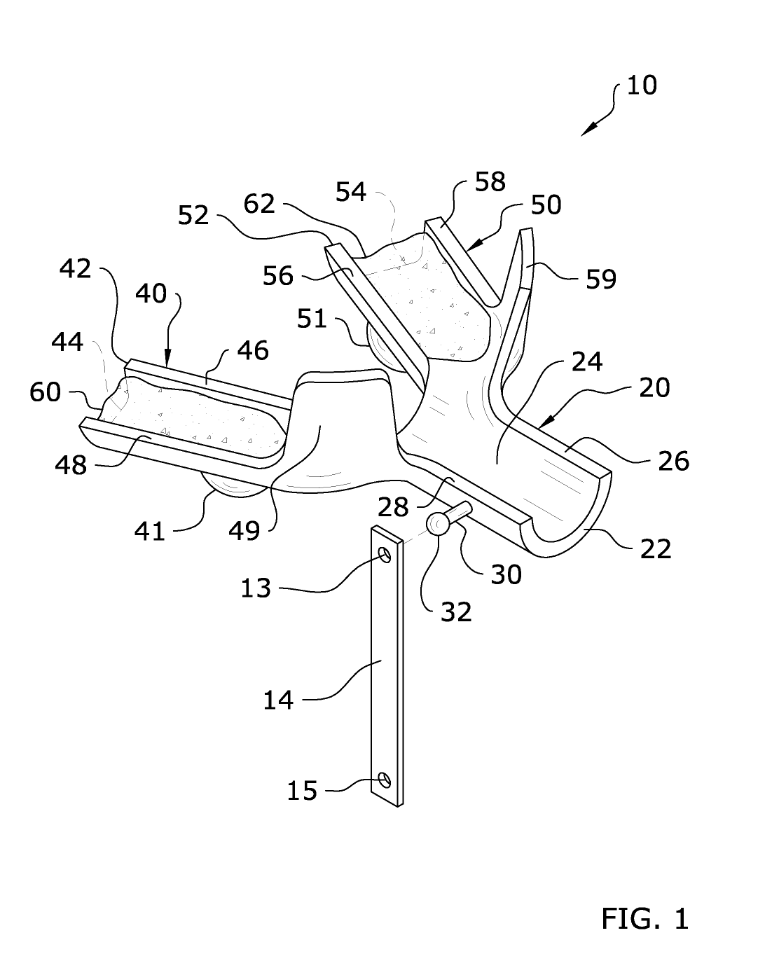

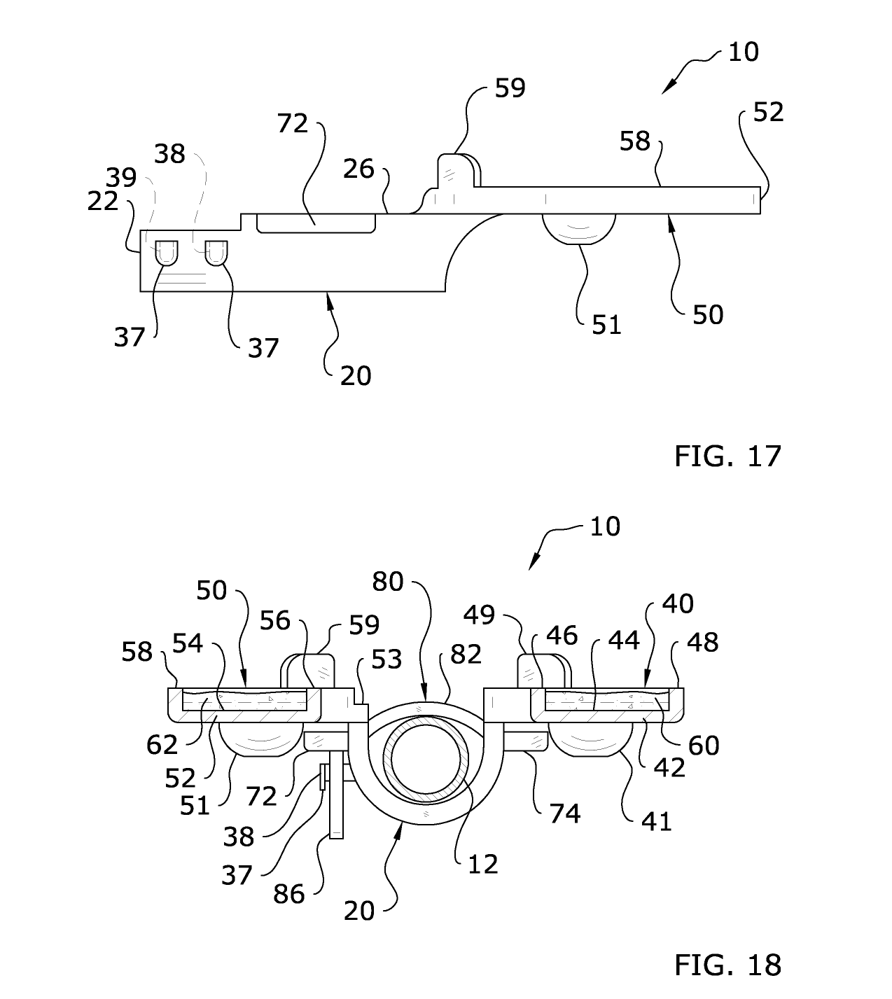

Turning now descriptively to the drawings, in which similar reference characters denote similar elements throughout the several views, FIGS. 1 through 19 illustrate an intubation tube oral appliance system 10, which comprises a tube support 20, a first tray 40 extending from the tube support 20 and a second tray 50 extending from the tube support 20. The tube support 20 is formed to receive an intubation tube 12 and a strap 14 secures the intubation tube 12 to the tube support 20. The first tray 40 and the second tray 50 include a thermoplastic layer that is formed to the ventilation patient's teeth. An antibacterial solution and/or fluoride solution is placed within the first tray 40 and second tray 50 prior to insertion into the ventilation patient's mouth to reduce dental plaque and VAP. The tube support 20, first tray 40 and the second tray 50 are preferably comprised of a unitary, hard material such as but not limited to acrylic.

### B. Tube Support

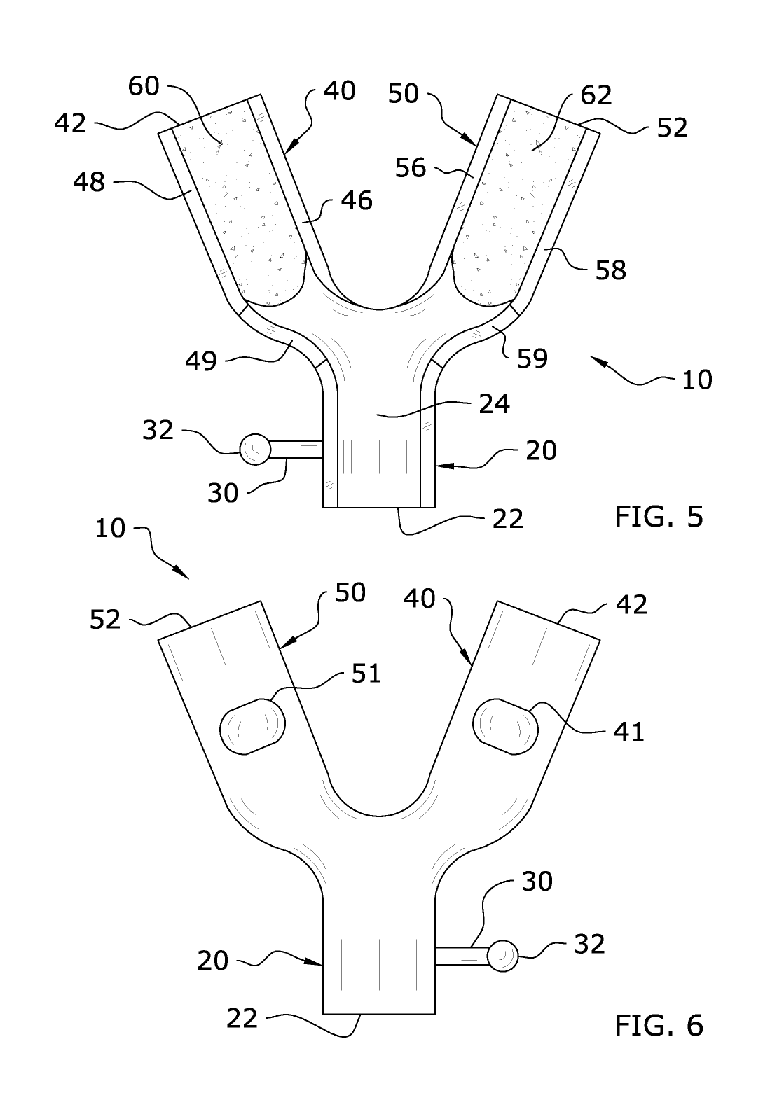

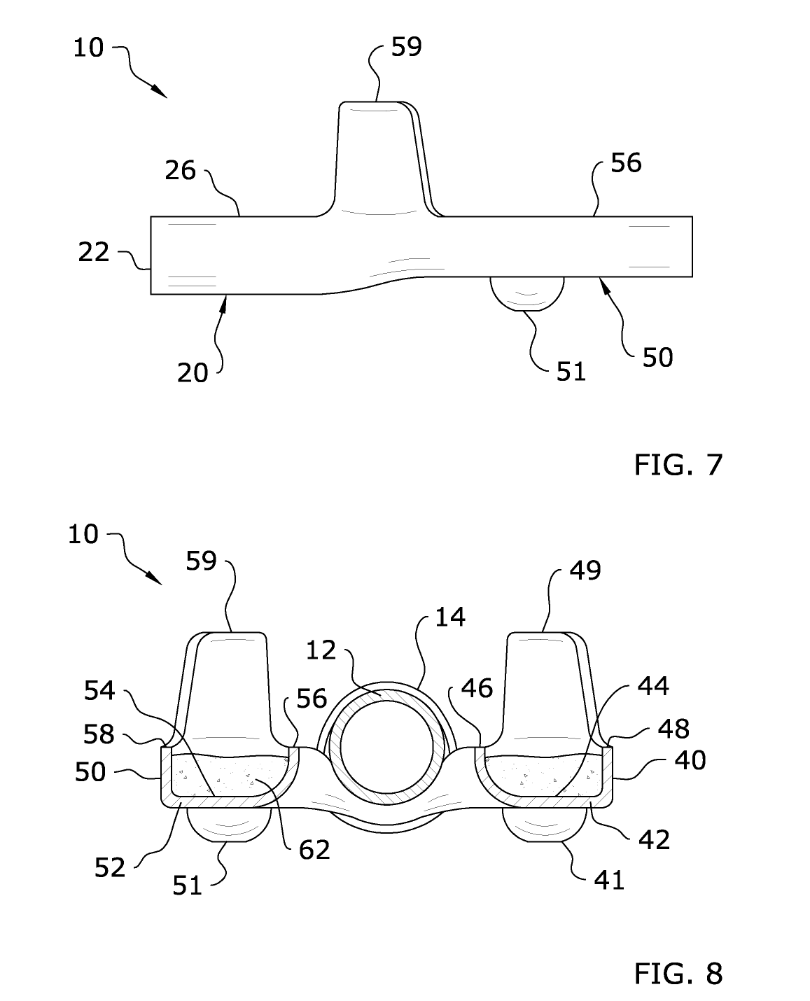

FIGS. 1 and 5 best illustrate the tube support 20 that forms a center channel 24 to receive a portion of an intubation tube 12 inserted into a ventilation patient. The tube support 20 is comprised of an elongated structure and is preferably straight as best illustrated in FIGS. 5 and 7 of the drawings.

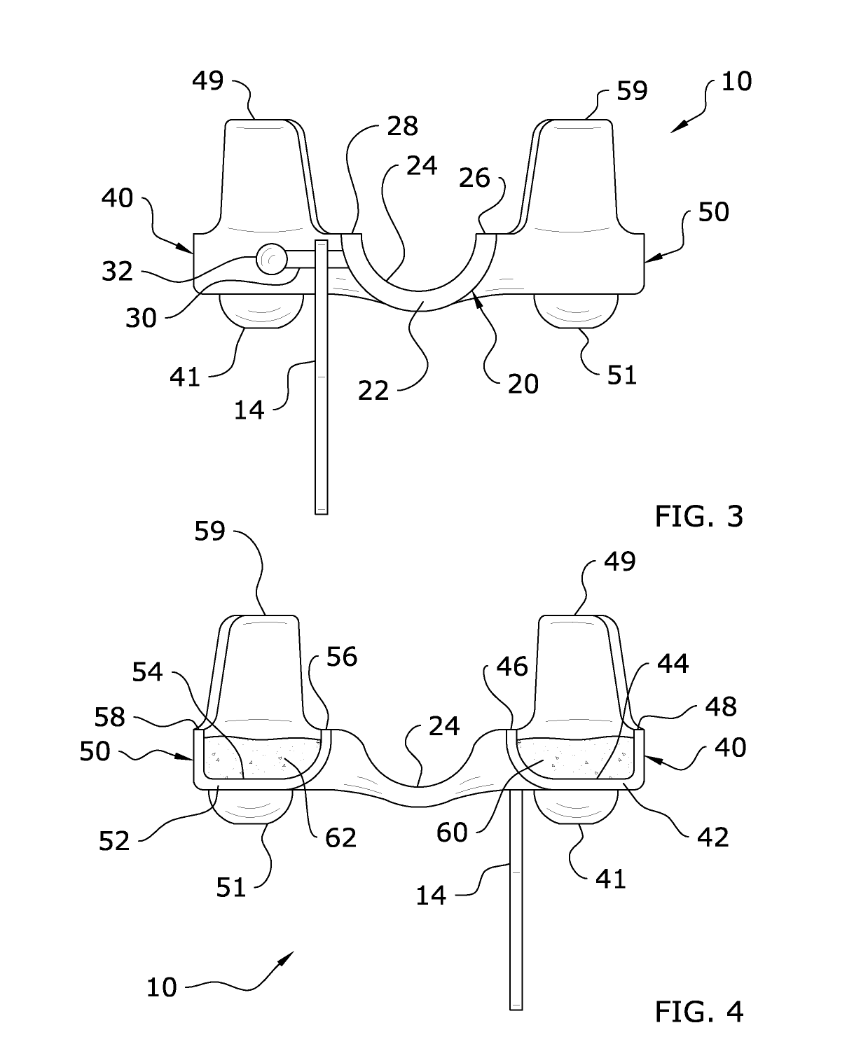

The tube support 20 includes an outer end 22 that extends outwardly from the ventilation patient's mouth. The tube support 20 further is preferably comprised of a first tube wall 26 and a second tube wall 28 that are substantially mirrors of one another as illustrated in FIGS. 1 and 3 of the drawings. The center channel 24 faces upwardly thereby allowing the intubation tube 12 to be positioned within and supported by the tube support 20.

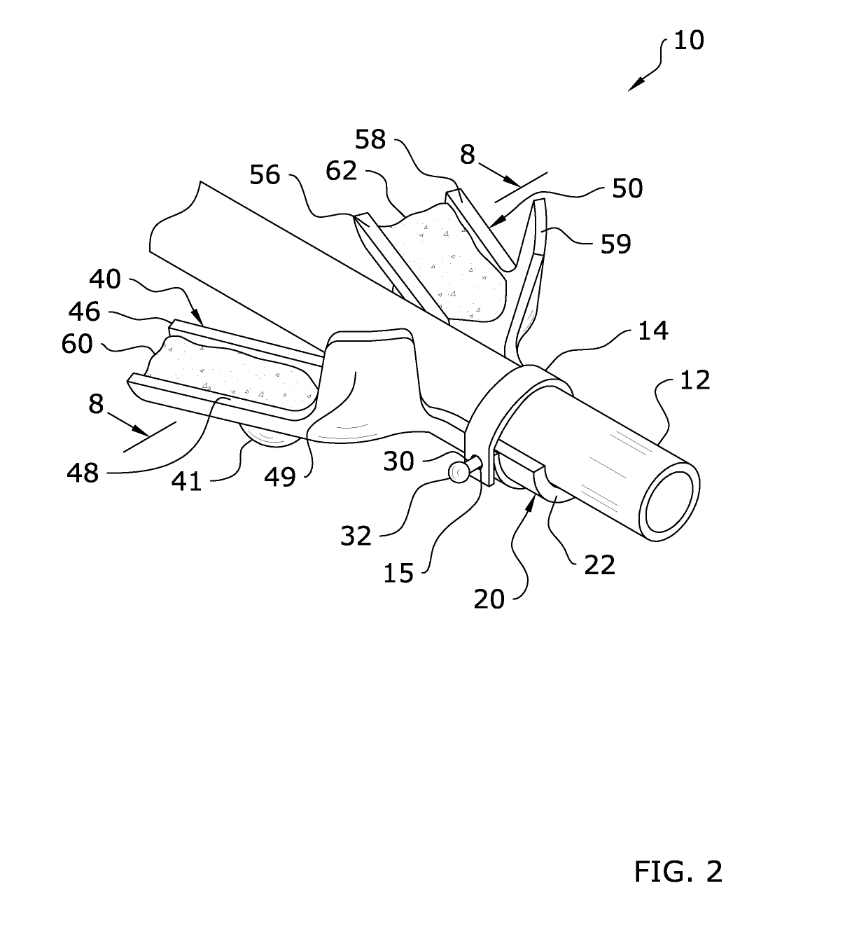

The tube support 20 further preferably has a U-shaped cross sectional shape forming the center channel 24 that receives a lower portion of the intubation tube 12 as illustrated in FIGS. 2 and 8 of the drawings. The center channel 24 has a width sufficient to receive the intubation tube 12 and preferably has a width approximately equal to the outer diameter of the intubation tube 12 to ensure a relatively secure fit of the intubation tube 12 within the center channel 24 as illustrated in FIG. 8 of the drawings.

### C. Trays

The first tray 40 extends from the tube support 20 at a first angle and the second tray 50 extends from the tube support 20 opposite of the first tray 40 at a second angle as illustrated in FIGS. 1 through 6 of the drawings. As best illustrated in FIG. 5 of the drawings, the first tray 40 and the second tray 50 preferably extend away from one another and are angled so that the first tray 40 is adapted to receive the right upper teeth of a ventilation patient and the second tray 50 is adapted to receive the left upper teeth of the ventilation patient. The first tray 40 and the second tray 50 are preferably approximately 45 mm apart from one another between their respect first end 42 and second end 52. The first tray 40 and the second tray 50 preferably substantially mirror one another as best illustrated in FIG. 5 of the drawings.

The first tray 40 includes a first channel 44 and the second tray 50 includes a second channel 54, wherein the first channel 44 and the second channel 54 are preferably connected to the center channel 24 of the tube support 20 to form a substantially Y-shaped channel structure as illustrated in FIG. 5 of the drawings. The first channel 44 and the second channel 54 further preferably form a substantially U-shaped channel that receives the upper teeth of the ventilation patient.

The first tray 40 includes a first channel 44 defined by an inner first wall 46, an outer first wall 48, and a first end 42 that is substantially open wherein the first end 42 is opposite of the tube support 20 as illustrated in FIG. 5 of the drawings. The outer first wall 48 is preferably substantially perpendicular with respect to a floor of the first channel 44 as best illustrated in FIG. 4 of the drawings. The inner first wall 46 is preferably angled at approximately 20 degrees from the floor of the first channel 44. The floor of the first channel 44 is preferably approximately 15 mm in width between the outer first wall 48 and the inner first wall 46. As discussed previously, the first tray 40 is adapted to receive a plurality of right upper teeth of a patient within the first channel 44.

The first tray 40 preferably includes a first thermoplastic layer 60 within the first channel 44. The first thermoplastic layer 60 becomes malleable when heated (e.g. by hot water) thereby allowing forming to the teeth of the ventilation patient prior to usage. After the first thermoplastic layer 60 cools to a certain temperature, the first thermoplastic layer 60 is no longer substantially malleable and is capable of receiving the teeth of the ventilation patient in a substantially snug manner. The imprints of the teeth formed into the first thermoplastic layer 60 also are adapted to receive an antibacterial solution and/or fluoride solution to assist in the dental care of the ventilation patient without having to periodically brush the teeth of the ventilation patient every two hours.

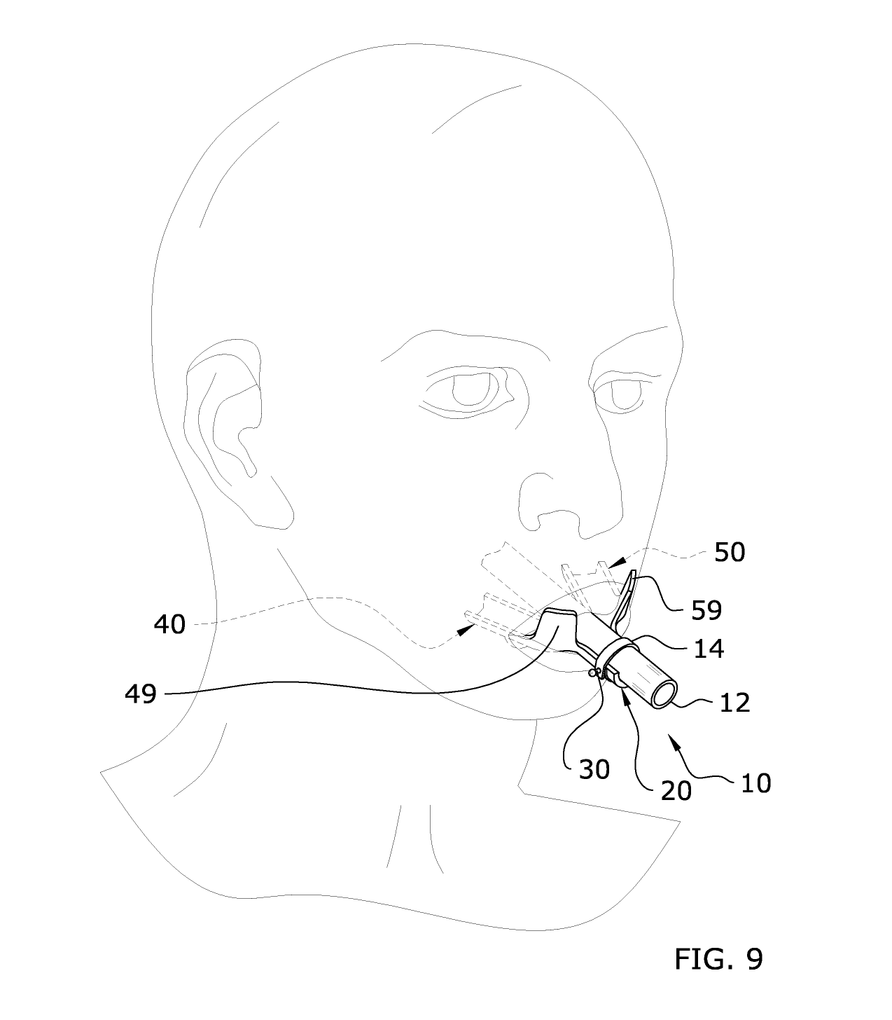

The first tray 40 further preferably includes a first flange 49 extending upwardly from a forward portion of the first tray 40. The first flange 49 is preferably slightly curved and is partially angled forwardly to comfortably remain outside of the mouth of the ventilation patient during usage as illustrated in FIG. 9 of the drawings. The first flange 49 preferably is approximately 20 mm in height and approximately 6 mm in width to adequately protect the upper lip during usage.

The first tray 40 further preferably includes a first spacer 41 extending downwardly from a lower surface of the first tray 40 as illustrated in FIGS. 1 through 4, 6 and 8 of the drawings. The first spacer 41 is preferably comprised of a rounded structure as best illustrated in FIGS. 4 and 6 of the drawings. The first spacer 41 provides spacing between the present invention and a conventional lower thermoplastic stock tray positioned upon the lower teeth of the ventilation patient. The first spacer 41 acts as a bite splint for the ventilation patient to bite upon and not damage the intubation tube 12.

The second tray 50 includes a second channel 54 defined by an inner second wall 56, an outer second wall 58, and a second end 52 that is substantially open wherein the second end 52 is opposite of the tube support 20 as illustrated in FIG. 5 of the drawings. The outer second wall 58 is preferably substantially perpendicular with respect to a floor of the second channel 54 as best illustrated in FIG. 4 of the drawings. The inner second wall 56 is preferably angled at approximately 20 degrees from the floor of the second channel 54. The floor of the second channel 54 is preferably approximately 15 mm in width between the outer second wall 58 and the inner second wall 56. As discussed previously, the second tray 50 is adapted to receive a plurality of right upper teeth of a patient within the second channel 54.

The second tray 50 preferably includes a second thermoplastic layer 62 within the second channel 54. The second thermoplastic layer 62 becomes malleable when heated (e.g. by hot water) thereby allowing forming to the teeth of the ventilation patient prior to usage. After the second thermoplastic layer 62 cools to a certain temperature, the second thermoplastic layer 62 is no longer substantially malleable and is capable of receiving the teeth of the ventilation patient in a substantially snug manner. The imprints of the teeth formed into the second thermoplastic layer 62 also are adapted to receive an antibacterial solution and/or fluoride solution to assist in the dental care of the ventilation patient without having to periodically brush the teeth of the ventilation patient every two hours.

The second tray 50 further preferably includes a second flange 59 extending upwardly from a forward portion of the second tray 50. The second flange 59 is preferably slightly curved and is partially angled forwardly to comfortably remain outside of the mouth of the ventilation patient during usage as illustrated in FIG. 9 of the drawings. The second flange 59 preferably is approximately 20 mm in height and approximately 6 mm in width to adequately protect the upper lip during usage.

The second tray 50 further preferably includes a second spacer 51 extending downwardly from a lower surface of the second tray 50 as illustrated in FIGS. 1 through 4, 6 and 8 of the drawings. The second spacer 51 is preferably comprised of a rounded structure as best illustrated in FIGS. 4 and 6 of the drawings. The second spacer 51 provides spacing between the present invention and a conventional lower thermoplastic stock tray positioned upon the lower teeth of the ventilation patient. The second spacer 51 acts as a bite splint for the ventilation patient to bite upon and not damage the intubation tube 12.

### D. Antibacterial Solution

The antibacterial solution may be comprised of a liquid or a gel. The antibacterial solution may further be comprised of any substance capable of killing bacteria such as but not limited to carbamide peroxide. The antibacterial solution may be stored and delivered via a syringe or related structure.

### E. Fluoride Solution

The fluoride solution may also be comprised of a liquid or gel. The fluoride solution may be comprised of any substance capable of strengthening teeth and limiting sensitivity of the teeth. The fluoride solution may also be stored and delivered via a syringe or related structure.

### F. Catch Member and Strap

FIGS. 1, 2, 3, 4 and 6 best illustrate a catch member 30 extending from the tube support 20. The catch member 30 is preferably comprised of an elongated structure that extends horizontally from either the first tube wall 26 or the second tube wall 28. The catch member 30 further preferably includes an end portion 32 that is broader than a width of the catch member 30 to catchably retain the strap 14 upon the catch member 30 when secured as illustrated in FIGS. 1 through 3. The end portion 32 preferably is comprised of a bulbous structure as illustrated in FIGS. 1 through 3.

The strap 14 is preferably comprised of an elastic material such as but not limited to rubber. The strap 14 includes a first aperture 13 and a second aperture 15 near an opposite end of the first aperture 13 as illustrated in FIG. 1 of the drawings. The first aperture 13 is positionable upon the catch member 30 initially and then the strap 14 is wrapped around the tube support 20 and the intubation tube 12 to retain the intubation tube 12 within the center channel 24. The second aperture 15 of the strap 14 is positioned over the catch member 30 to retain the strap 14 about the intubation tube 12 as illustrated in FIG. 2 of the drawings.

### G. First Alternative Embodiment

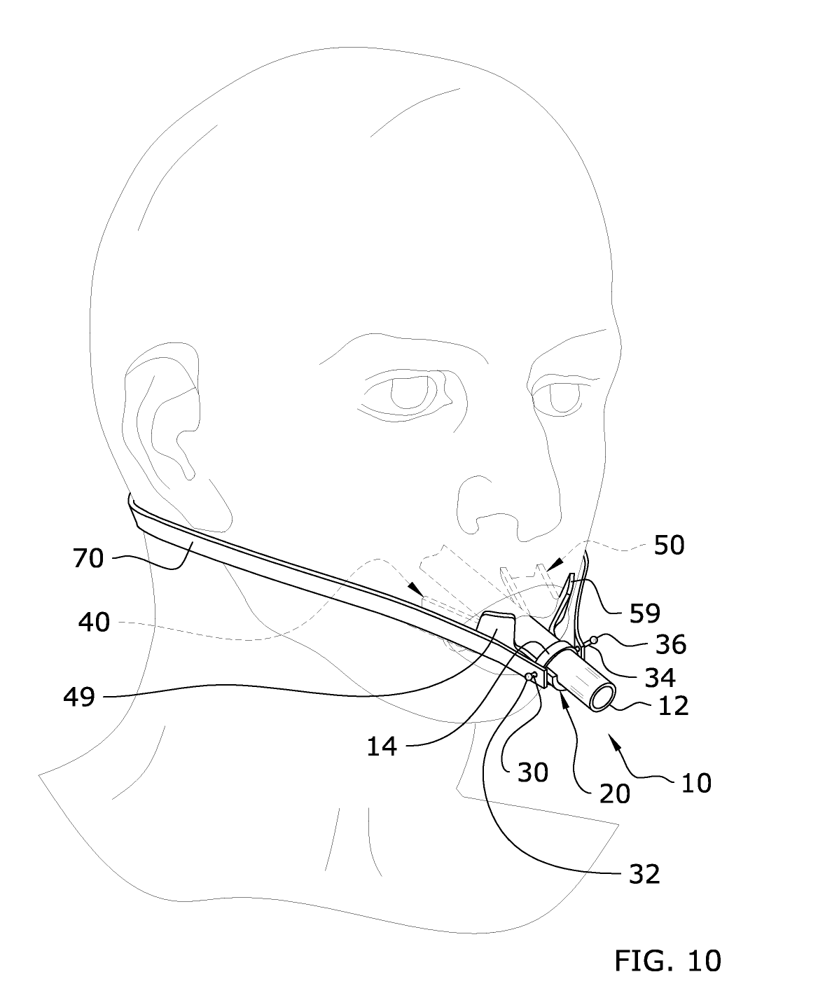

FIG. 10 illustrates a first alternative embodiment of the present invention comprised of a second catch member 34 extending from the tube support 20 opposite of the first catch member 30. The second catch member 34 includes a second end portion 36 similar to the first end portion 32 as shown in FIG. 10. The strap 14 is attached to the first catch member 30 and the second catch member 34 to retain the intubation tube 12 as illustrated in FIG. 10 of the drawings. In addition, a head strap 70 having distal apertures at opposing ends is attached to the first catch member 30 and the second catch member 34 to secure the present invention to the head of a patient.

### H. Second Alternative Embodiment

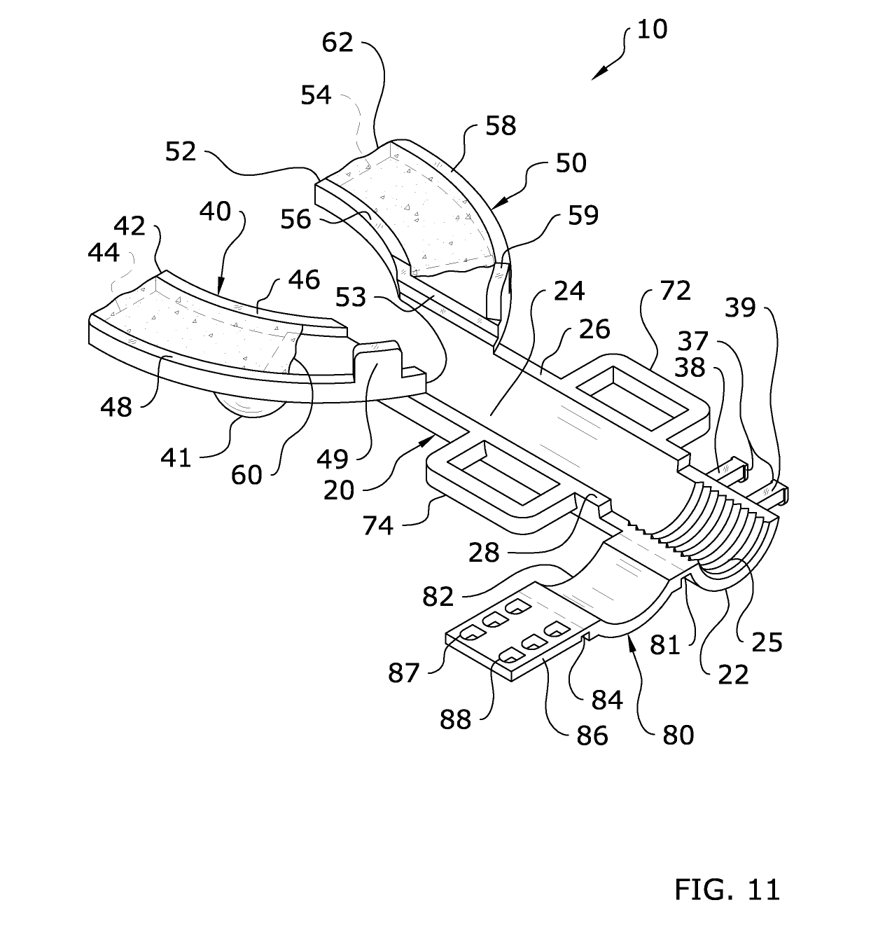

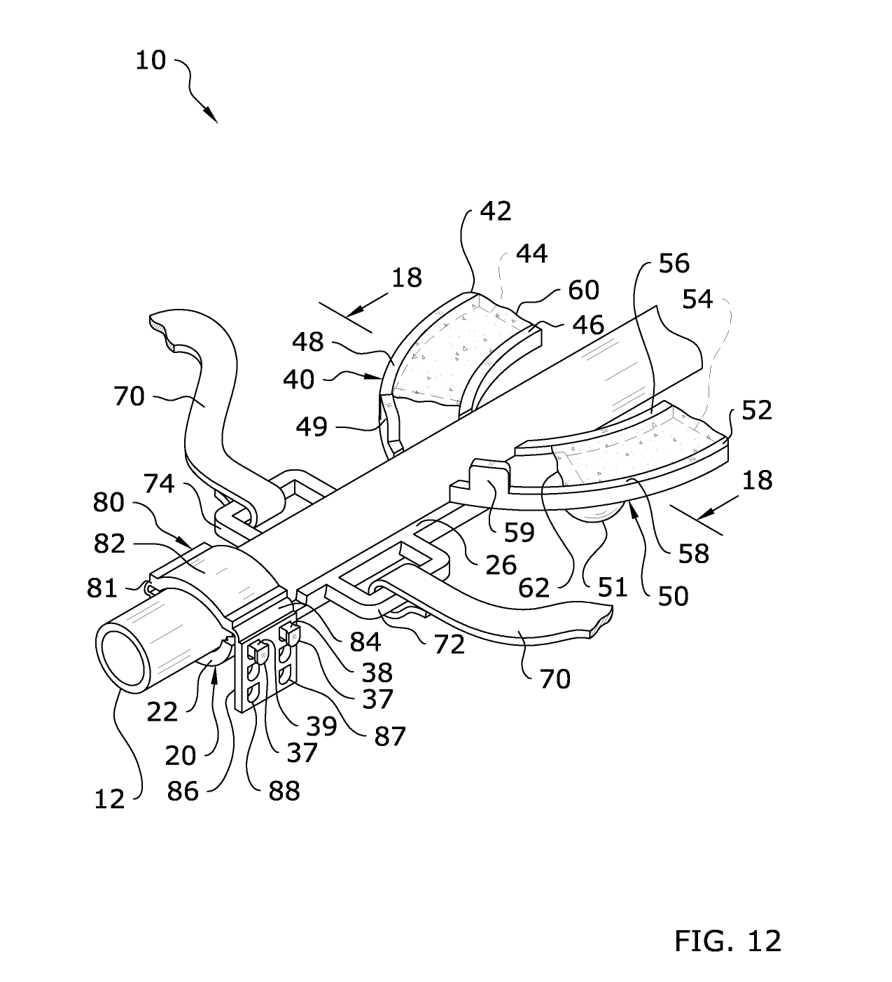

FIGS. 11 through 18 illustrate a second alternative embodiment of the present invention that has features and functions similar to the prior embodiments. As best illustrated in FIGS. 11 and 12, the second alternative embodiment includes a securing member 80, a gripping portion 25 within the center channel 24, a pair of connectors 72, 74 for receiving a head strap 70 and a side channel 53 for allowing ventilation along the side of the intubation tube 12.

i. Securing Member.

The securing member 80 selectively secures the intubation tube 12 instead of the strap 14 illustrated previously. The securing member 80 is preferably comprised of the same material as the tube support 20 and integrally formed with respect to the tube support 20. However, the securing member 80 may be comprised of a material different from the tube support 20 and attached to the tube support 20. It is preferable that the securing member 80 be comprised of a rigid or semi-rigid material (e.g. plastic) to increase the compression force upon the intubation tube 12.

As illustrated in FIG. 11 of the drawings, the securing member 80 is comprised of an elongated strap-like structure that extends outwardly from the side of the tube support 20. The securing member 80 is preferably adjacent to the outer end 22 of the tube support 20. The securing member 80 selectively engages one or more members 38, 39 on the opposite side of the tube support 20 as discussed further herein.

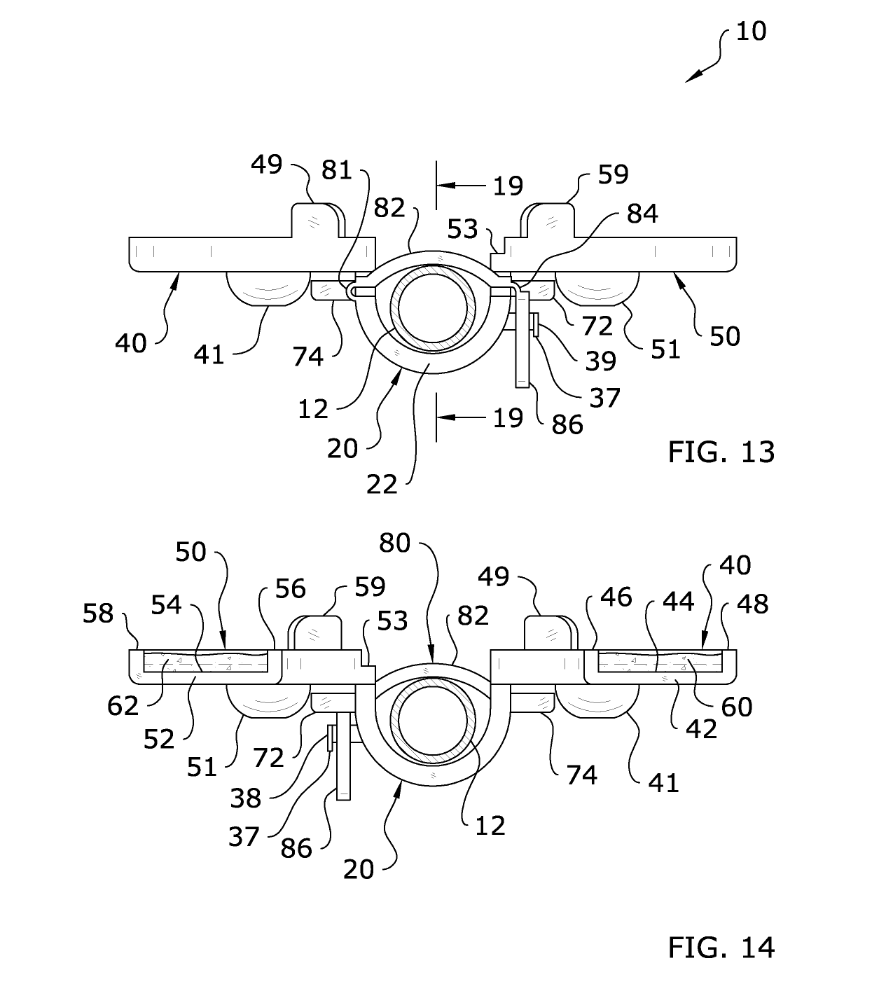

The securing member is comprised of a first hinge 81 that is attached to the upper end of the side of the tube support 20. FIGS. 11 and 13 best illustrate the preferred connection of the first hinge 81 to the tube support 20.

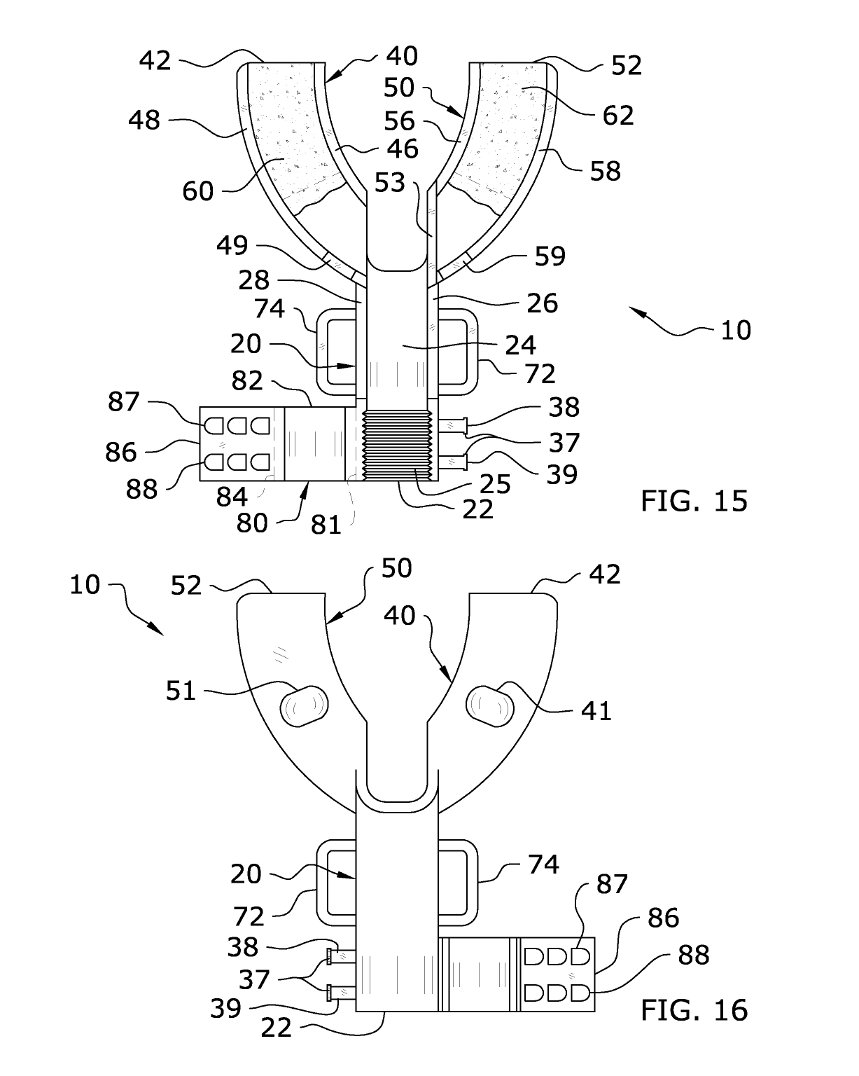

An engaging portion 82 is pivotally connected to the tube support 20. The engaging portion 82 extends outwardly from the first hinge 81 as further shown in FIGS. 11 and 13. The engaging portion 82 is preferably rigid and significantly thicker than the first hinge 81 as illustrated in FIG. 13 of the drawings. The width of the engaging portion 82 is preferably approximately the same as the distance the gripping portion 25 extends inwardly as best illustrate in FIG. 15. The width of the engaging portion 82 is preferably at least 9 mm.

The engaging portion 82 is preferably comprised of a curved shape as further illustrated in FIG. 13 of the drawings. The interior surface of the engaging portion 82 preferably has a significantly larger radius than the center channel 24 to ensure increased compression of the intubation tube 12 when the securing member 80 is in the closed position as illustrated in FIG. 13 of the drawings. It is preferable that the distance between the center of the interior surface to the center of the center channel 24 is at least 8.5 mm to ensure sufficient clamping force upon a 9.66 mm tube.

FIGS. 11 and 13 further illustrate a securing flap 86 connected to the engaging portion 82 opposite of the first hinge 81. The securing flap 86 is further preferably comprised of a rigid material and has a thickness greater than the first hinge 81. The securing flap 86 is preferably comprised of a planar structure as illustrated in FIGS. 11, 12 and 13 of the drawings. The securing flap 86 is pivotally attached to the engaging portion 82 by a second hinge 84. The first hinge 81 and the second hinge 84 are preferably similar in structure and are preferably comprised of a thinner material than the engaging portion 82.

One or more first apertures 87 extend through the securing flap 86 that are parallel to one or more second apertures 88 as illustrated in FIGS. 11, 15, 16 of the drawings. The first apertures 87 and the second apertures 88 catchably receive a first member 38 and a second member 39 respectively that extend outwardly from an opposite side of the tube support 20 as illustrated in FIG. 12 of the drawings. The first member 38 and the second member 39 extend outwardly and each preferably include a catch flange 37 that extends outwardly from a distal end thereof as illustrated in FIGS. 11 and 12 of the drawings. The catch flange 37 preferably has a U-shape and is larger than the apertures 87, 88 to prevent accidental removal of the securing flap 86 from the members 38, 39 as shown in FIG. 12 of the drawings. It is further preferable that the apertures 87, 88 each preferably have a U-shaped structure as best illustrated in FIGS. 12 and 15 of the drawings.

It is further preferable to have two or more rows of apertures 87, 88 to provide for multiple positions depending upon the size of the intubation tube 12 as illustrated in FIGS. 11 and 15. For example, if the intubation tube 12 has a larger diameter, the user would secure the securing flap 86 with the outer row of apertures. If the intubation tube 12 has a smaller diameter, the user would secure the securing flap 86 with the inner row apertures as illustrated in FIG. 12 of the drawings. Any intubation tubes 12 having a diameter between thereof can be secured with the middle row of apertures. While a pair of apertures 87, 88 preferably form each row for increasing the stability of the securing member 80, it can be appreciated that a single aperture may form each row or more than two apertures may form each row.

In operation of the securing member 80, the intubation tube 12 is positioned within the center channel 24 of the tube support 20 as illustrated in FIG. 12 of the drawings. FIG. 11 illustrates the securing member 80 in the open position to allow for positioning of the intubation tube 12 within the center channel. After the intubation tube 12 is properly positioned, the user then pivots the engaging portion 82 over the top of the intubation tube 12 and pivots the securing flap 86 to catchably engage the members 38, 39 within corresponding apertures 87, 88 to engage the intubation tube 12 in a compression force manner that prevents movement of the intubation tube 12 within the tube support 20. FIGS. 12, 13, 14 and 18 illustrate the securing member 80 in the closed position securing the intubation tube 12 within the tube support 20.

ii. Gripping Portion.

The center channel 24 of the tube support 20 includes a gripping portion 25 that is opposite of the securing member 80 when the securing member 80 is in the closed position. The gripping portion 25 is adapted to engage the intubation tube 12 to prevent longitudinal movement of the intubation tube 12 within the tube support 20. The gripping portion 25 is preferably formed within the tube support 20 but may also be attached to the tube support 20. The gripping portion 25 is preferably near or adjacent to the outer end 22 of the tube support 20.

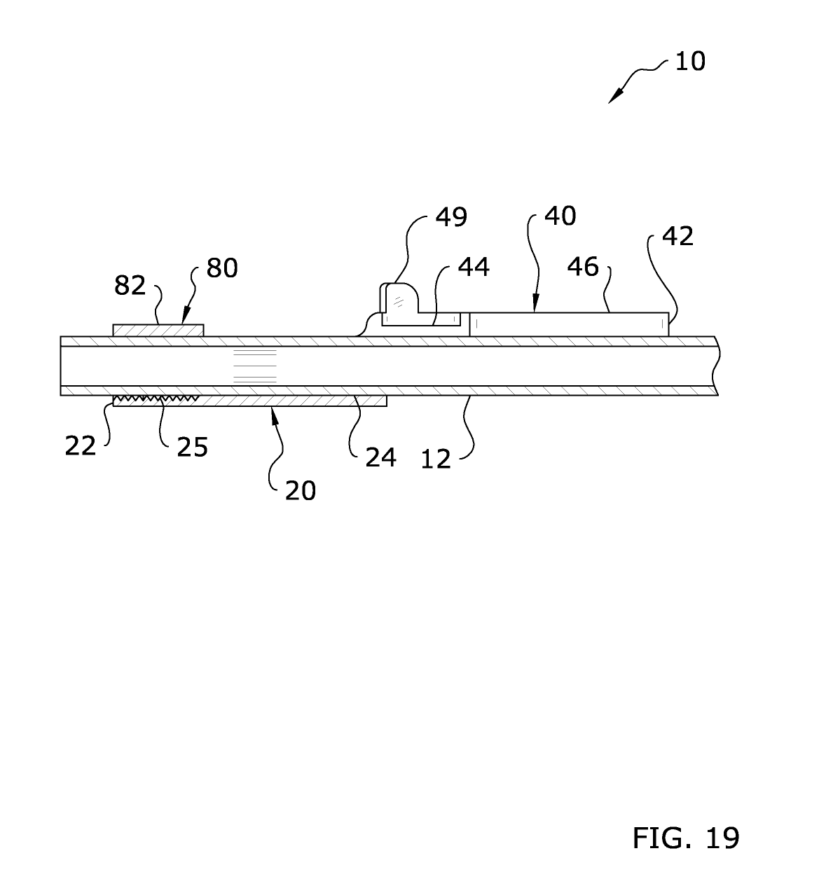

As best illustrated in FIGS. 11, 15 and 19 of the drawings, the gripping portion 25 is preferably comprised of a plurality of ridges. The plurality of ridges are preferably parallel to one another as illustrated in FIG. 15. The upper edge of the plurality of ridges may extend above the surface of the center channel 24, at the same level as the surface of the center channel 24 or below the surface of the center channel 24. The upper edge for each of the ridges is preferably pointed to engage the intubation tube 12 as best illustrated in FIG. 19. The gripping portion 25 preferably extends between both upper edges of the tube support 20 as illustrated in FIG. 11 of the drawings.

As illustrated in FIG. 19, the ridges forming the gripping portion 25 preferably are tapered in an inverted V-shaped structure. It is preferable that the angle of the V-shaped grooves formed between the ridges is approximately 60 degrees and that the depth of the grooves is approximately 0.90 mm wherein the thickness of the tube support 20 is at least 2 mm. The gripping portion 25 preferably extends inwardly from the outer end 22 a distance of at least 9 mm. The plurality of ridges for the gripping portion 25 are perpendicular with respect to a longitudinal axis of the tube support 20.

iii. Connectors.

FIGS. 11, 12, 15 and 16 best illustrate a first connector 72 extending outwardly from the first tube wall 26 and a second connector 74 extending outwardly from the first tube wall 28. The connectors 72, 74 preferably are integrally formed within the tube support 20, however, the connectors 72, 74 may be attached to the tube support 20. The connectors 72, 74 each form an opening that receives an end of the head strap 70 used to secure the present invention to the user's head. The connectors 72, 74 preferably extend outwardly away from one another along a common plane. The connectors 72, 74 each have a U-shaped structure as further illustrated in FIGS. 11 and 15 of the drawings.

iv. Side Channel.

FIGS. 1 and 15 illustrate the side channel 53. The side channel 53 extends into the second tray 50 adjacent to the center channel 24 to allow for providing a gap between the intubation tube 12 and the second tray 50 for the pilot tube. The pilot tube is an access tube that comes out of the side of the endotracheal tube that is used for suctioning subglottic secretions that accumulate on top of the balloon that occluded the airway. The side channel 53 prevents blocking of the pilot tube.

### I. Operation of Invention.

In use, the first thermoplastic layer 60 and the second thermoplastic layer 62 are heated to a state where they become malleable (e.g. by placing in hot water). Once the first thermoplastic layer 60 and the second thermoplastic layer 62 are malleable, the trays 40, 50 are positioned upon the ventilation patient's upper teeth to be fitted to the patient's upper teeth by forming an imprint of the upper teeth in the first thermoplastic layer 60 and the second thermoplastic layer 62. The trays 40, 50 are removed from the upper teeth and the first thermoplastic layer 60 and the second thermoplastic layer 62 are allowed to cool into a hardened state.

The antibacterial solution is then placed on the first thermoplastic layer 60 and the second thermoplastic layer 62 prior to insertion into the mouth of the ventilation patient. The fluoride solution may be utilized after 12 hours of the antibacterial solution to rotate the solutions used. After the antibacterial solution is placed upon the first thermoplastic layer 60 and the second thermoplastic layer 62, the trays 40, 50 are reinserted into the ventilation patient's mouth with the first flange 49 and the second flange 59 remaining outside of the upper lip to protect the upper lip as illustrated in FIG. 9 of the drawings. In addition, the tube support 20 is positioned near and in front of the teeth of the patient outside of the oral cavity.

The intubation tube 12 is then positioned within the center channel 24 of the tube support 20 and the strap 14 (or securing member 80) is then secured to retain the intubation tube 12 in a desired position. After a period of time (e.g. 12 hours), the present invention may be removed, cleaned and a replacement solution (e.g. antibacterial or fluoride) applied to the first thermoplastic layer 60 and the second thermoplastic layer 62. It can be appreciated that a conventional basic thermoplastic stock tray may be attached to the lower teeth of the patient opposite of the present invention.

Unless otherwise defined, all technical and scientific terms used herein have the same meaning as commonly understood by one of ordinary skill in the art to which this invention belongs. Although methods and materials similar to or equivalent to those described herein can be used in the practice or testing of the present invention, suitable methods and materials are described above. All publications, patent applications, patents, and other references mentioned herein are incorporated by reference in their entirety to the extent allowed by applicable law and regulations. In case of conflict, the present specification, including definitions, will control. The present invention may be embodied in other specific forms without departing from the spirit or essential attributes thereof, and it is therefore desired that the present embodiment be considered in all respects as illustrative and not restrictive. Any headings utilized within the description are for convenience only and have no legal or limiting effect.

The invention claimed is:

1. An intubation tube oral appliance, comprising:
a tube support forming a center channel adapted to receive an intubation tube;
a first tray extending from said tube support at a first angle, wherein said first tray is adapted to receive a plurality of right upper teeth of a patient;
a second tray extending from said tube support at a second angle, wherein said second tray is adapted to receive a plurality of left upper teeth of a patient; and
a securing member attached to said tube support, wherein said securing member has a closed position to secure the intubation tube within said center channel and an open position to allow for positioning of the intubation tube within said center channel.

2. The intubation tube oral appliance of claim 1, including a first thermoplastic layer within said first tray and a second thermoplastic layer within said second tray.

3. The intubation tube oral appliance of claim 1, including a volume of antibacterial solution within said first tray and said second tray.

4. The intubation tube oral appliance of claim 1, wherein said first tray includes a first channel defined by an inner first wall and an outer first wall, wherein said second tray includes a second channel defined by an inner second wall and an outer second wall.

5. The intubation tube oral appliance of claim 4, wherein said first tray includes a first end that is substantially open and wherein said second tray includes a second end that is substantially open.

6. The intubation tube oral appliance of claim 1, wherein said securing member is comprised of an engaging portion pivotally attached to said tube support.

7. The intubation tube oral appliance of claim 6, wherein said securing member is comprised of a securing flap pivotally attached to said engaging portion opposite of said tube support.

8. The intubation tube oral appliance of claim 7, including one or more members extending from said tube support, and wherein said securing flap includes one more apertures that catchably receive said one or more members.

9. The intubation tube oral appliance of claim 8, wherein said securing flap includes two or more rows of apertures that receive said one or more members.

10. The intubation tube oral appliance of claim 6, wherein said engaging portion is comprised of a curved structure.

11. The intubation tube oral appliance of claim 6, wherein said engaging portion is comprised of a rigid structure.

12. The intubation tube oral appliance of claim 11, wherein said engaging portion is pivotally attached to said tube support by a first hinge.

13. The intubation tube oral appliance of claim 1, including a gripping portion within said center channel near an outer end of said center channel.

14. The intubation tube oral appliance of claim 13, wherein said gripping portion is comprised of a plurality of ridges that are perpendicular with respect to a longitudinal axis of said tube support.

15. The intubation tube oral appliance of claim 14, wherein said plurality of ridges are each comprised of an inverted V-shaped structure.

16. The intubation tube oral appliance of claim 13, wherein said gripping portion is adjacent to said outer end.

17. An intubation tube oral appliance, comprising:
a tube support forming a center channel;
a first tray extending from said tube support at a first angle, wherein said first tray is adapted to receive a plurality of right upper teeth of a patient;
a second tray extending from said tube support at a second angle, wherein said second tray is adapted to receive a plurality of left upper teeth of a patient; and
a securing member attached to said tube support; and
an intubation tube positioned within said center channel of said tube support;
wherein said securing member has a closed position to secure the intubation tube within said center channel and an open position to allow for positioning of the intubation tube within said center channel

18. The intubation tube oral appliance of claim 17, wherein said securing member is comprised of an engaging portion pivotally attached to said tube support.

19. The intubation tube oral appliance of claim 18, wherein said securing member is comprised of a securing flap pivotally attached to said engaging portion opposite of said tube support.

20. An intubation tube oral appliance, comprising:
a tube support forming a center channel adapted to receive an intubation tube;
a first tray extending from said tube support at a first angle, wherein said first tray is adapted to receive a plurality of right upper teeth of a patient;
a second tray extending from said tube support at a second angle, wherein said second tray is adapted to receive a plurality of left upper teeth of a patient;
a securing member attached to said tube support, wherein said securing member has a closed position to secure the intubation tube within said center channel and an open position to allow for positioning of the intubation tube within said center channel;
wherein said securing member is comprised of an engaging portion pivotally attached to said tube support and a securing flap pivotally attached to said engaging portion opposite of said tube support;
one or more members extending from said tube support, and wherein said securing flap includes one more apertures that catchably receive said one or more members; and
a gripping portion within said center channel near an outer end of said center channel and aligned with said securing member.
